(19) United States
(12) Patent Application Publication
(10) Pub. No.: US 2003/0158204 A1
(43) Pub. Date: Aug. 21, 2003

(54) Diamino phenothiazine derivatives and composition comprising same

(76) Inventor: Laurent Galey, Emerainville (FR)

Correspondence Address: YOUNG & THOMPSON, 745 SOUTH 23RD STREET 2ND FLOOR, ARLINGTON, VA, 22202

(21) Appl. No.: 10/311,005

(22) PCT Filed: Jun. 15, 2001
(86) PCT No.: PCT/FR01/01888

(30) Foreign Application Priority Data
Jun. 15, 2000 (FR) 00/07660

(51) Int. Cl. A61K 31/498; C07D 241/46

(52) U.S. Cl. 514/250; 544/347

(57) ABSTRACT

The invention concerns a diamino phenothiazine derivative, at least monosubstituted in one of its positions 1, 2, 3, 4, 5, 6, 7 or 8 and having a quinone para and ortho tautomerism, of general formula (I) or one of its salts, wherein: R1, R2, R4, R5, R6, R7, R8 and R1′, R2′, R3′, R4′ are identical or different from one another and represent: a hydrogen atom, an aliphatic and/or aromatic and/or cyclic radical, such as an alkyl group, an alkenyl group, an alkynyl group, a hydroxy group, an aryloxy group, a ketone group, an amide group, a carboxy group, a base, a non-organic radical such as a halogen atom, an alkaline-earth, a metal atom; and X is a mineral or organic anion, except for blue A, blue B, blue C, methylene green, novel methylene blue, toluidine blue derivatives. The invention is applicable in the biological and/or chemical field.

The present invention relates to organic and/or mineral derivatives of phenothiazine useful in the field of general chemistry and therapeutics in applications such as human and veterinary medicine, the agro-food field, the treatment of waters and general physico-chemistry, as well as compositions with biological and/or chemical activity which comprise organic and/or mineral derivatives of phenothiazine, useful in the field of general chemistry and therapeutics in applications such as human and veterinary medicine, the agro-food field, the treatment of waters and general physico-chemistry.

In the 1880s, the first colored phenothiazines were synthesized, with a diamino phenothiazine structure of which the most famous was methylene blue. A large part of these phenothiazines was or is still used in histology (azures, toluidine blue, etc.).

Methylene blue has a moderate “antiseptic” activity, used externally but also internally, which caused it to be included in numerous pharmacopias for nearly a century. The first medical applications date from this period, and won for Paul Ehrlich the Nobel Prize in medicine in the 1920s. He postulated the specific coloration of an organ or a biological structure indicated the existence of a reversible colorant-biological structure interaction, which led to the ligand-receptor theory.

Another activity, the anti-parasitic one, was studied toward this era, against the Plasmodium type, hematozoic epidemic over almost all of the tropical and subtropical regions, and which also involved several billion human beings (cf. Guttmann P., Ehrlich P. On the Effect of Methylene Blue on Malaria, Berliner klinische Wochenschrift 1891 39 :September 28.)

This malady, transmitted by hematophagic mosquitoes, had a complex clinical table, dominated by chronic blood pancytopeny, research, and of which the mortality-morbidity is immeasurable in the affected countries. Above all, there exists a serious form of the malady, due to Plasmodium falciparum, with pernicious or neuropaludic access, which consists in grave encephalopathy and various symptoms (coma, stupor, convulsions, paresthesis, paralysis, etc.).

The mortality is of the order of 50%, the percentage of neurological series being 20%; there is thus a medical urgency, whilst the treatment remains the same for more than one century, and consists almost exclusively in the administration of quinine salts.

The mechanism of this acute fibrile encephalopathy commences by being comprised, and is almost another medical emergency, toxic shock.

During pernicious attack and toxic shock, there exists an excess of production of TNF (Tumor Necrosis Factor) and pro-inflammatory cytokines (interleukins and interferons) in the sick people in the acute phase, (cf. Shaffer N, Grau G E, Hedberg K, Davachi F, Lyamba B, Bightower A W, Breman J G, Phuc N D Tumor necrosis factor and severe malaria.J Infect Dis 1991 January;163(l):96-101 and Chuncharunee S, Jootar 5, Leelasiri A, Archararit N, Prayoonwiwat W, Mongkonsritragoon W, Polvicha P, Srichaikul T, Levels of serum tumor necrosis factor alpha in relation to clinical involvement and treatment among Thai adults with Plasmodium falciparum malaria.J Med Assoc Thai 1997 September;80 Suppi 1:572-5 Wattavidanage J, et al, TNFalpha*2 marks high risk of severe disease during Plasmodium falciparum malaria and other infections in Sri Lankans. din Exp Immunol. 1999 February;115(2):350-5.).

These parasitic and/or microbiological shock conditions lead to hyperproduction of the pro-inflammatory factors, which lead to the massive release by the host cells of cytotoxic effectors, of the free radical type, giving rise to a shock condition. These cytotoxic effector free radicals are radicals derived from oxygen (superoxide, hydroxyl, etc.) and nitrogen (nitric oxide, peroxynitrite). A abnormal quantity of nitrites is at present encountered in sick people with pernicious attack, (cf. Senaldi G, et al. Nitric oxide and cerebral malaria. Lancet. 1992 December 19-26;340(8834-8835):1554 ; Mulder B, et al, The role of nitric oxide in cerebral malaria, Med Trop (Mars). 1995;55(4 Suppl):114-5 ; Moskowitz M A, et al., Nitric oxide and cerebral ischemia, Adv Neurol. 199e;71:365-7; discussion 367-9. Anstey N M, et al, Nitric oxide in Tanzanian children with malaria: inverse relationship between malaria severity and nitric oxide production/nitric oxide synthetase type 2 expression, J Exp Med. 1996 August 1;184(2) :557-67).

This type of clinical table other than pathology, is found in toxic shocks, particularly with Gram negative bacteria. There is noted the condition of stupor, hypertension, nervous troubles, fever. Upon autopsy, there are discovered pancytopenie, congestion of the spleen, of the kidneys, of the SNC, rosaceia (liver, brain, peritoneum), intestinal, pulmonary hemorrhages, etc.

Methylene blue is active by the IV route, on pernicious attacks resistant to quinine, (cf. Couto M., Endo-venous injections of methylene blue in malaria, Bulletin of the society of exotic pathology 1908 I, 4 :292-295.)

This indication was however rapidly abandoned, as it became well known, for reasons hardly comprehensible, due perhaps to the lack of reliability of the product, which intensely colors the urine and feces or else because it becomes very quickly ineffective as a disinfection agent.

The use of other synthetic anti-malarials (amino-S and amino-4-quinolene) caused this indication to be forgotten, even though very interesting. The product itself is inexpensive, has a high therapeutic coefficient, is usable by all enteral and parenteral routes. Above all, it is almost not toxic, and is contraindicated only in rare cases (G6PD deficit for example).

From these observations, the present inventor has paid attention to the development of a family of derivatives of diamino-phenothiazine structure and other associated phenothiazines (cf. (cf. Cub F, Sabobovic D, Somogyi L, Marusic M, Berbiguier N, Galey L : Anti-tumoral and anti-inflammatory effects of biological stains, Agents Actions 1991 November;34(3-4):424-8), having a fourfold activity similar to that observed for methylene blue, namely:

Anti-tumoral, anti-inflammatory, anti-endotoxic shock and anti-toxic activity, by non-specific terminal inhibition of the cytotoxic agents of specific or unspecific immunological or immunopathological reactions.

An anti-bacterial, anti-viral and anti-parasitic activity according to the properties described in 1) as well as by direct action on the metabolism, and the genetic of the pathogenic organisms, and an effect of the poison respiratory type,

A direct activity on the nucleic acids with directed specific reversible and topological liaison effect

Powerful antioxidant activity, as to cytotoxic free radicals and/or mutagenic products of metabolism, the intoxications or irradiations and permitting its use in these circumstances in acute and/or chronic phases, accidental or provoked, due to these same radicals.

Certain derivatives of diamino-phenothiazines are known, for example, to detect the presence of a reducing agent such as ascorbic acid as is described in GB-A-2 002 517. They thus serve in analysis devices to determine the presence of this reducing agent in a liquid specimen.

In EP-A-510 668 are described phenothiazine derivatives with a diamino-phenothiazine structure applicable to photodynamic therapy of cancer or of the immunoessays using chemoluminescence.

In WO-A-9925388, there is proposed a new derivative of toluidine which permits the detection of suspect dysplastic tissues, in particular cancerous or pre-cancerous tissues.

The known derivatives of phenothiazine with a diamino-phenothiazine structure are thus principally known as agents for detection and/or reaction and not as therapeutic agents.

The present invention thus has for its first object to provide new organic and/or mineral derivatives of diamino-phenothiazine and other associated phenothiazines having physico-chemical and biological activities whose potential effects are usable in the field of agro-food, biology and physical chemistry as well as in human and veterinary medicine.

To this end, the invention has for its object a diamino-phenothiazine derivative, at least monosubstituted at one of the 1, 2, 3, 4, 5, 6, 7 or 8 positions and having a para and ortho quinonic tautometry, of general formula (I) or one of its salts,

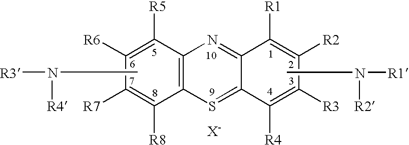

in which

R1, R2, R4, R5, R6, R7, R8 and R1′, R2′, R3′ R4′ are identical or different from each other and represent:

a hydrogen atom, an aliphatic and/or aromatic and/or cyclic radical, such as

an alkyl group, a alkenyl group, an alkynyl group, a hydroxy group, an aryloxy group, a ketone group, an amide group, a carboxy group, a base, etc., an inorganic radical such as a halogen atom, an alkaline earth, a metallic atom and X is a mineral or organic anion,

except for derivatives that are azure A, azure B, azure C, methylene green, the new methylene blue, toluidine blue.

The present invention also has for its object providing a composition with biological and/or chemical activity comprising at least one organic and/or mineral derivative of diamino-phenothiazine and other associated phenothiazines having physico-chemical and biological activities whose potential effects are usable in the field of argo-food, biology and physical chemistry as well as in human and veterinary medicine.

To this end, the invention thus also has for its object a composition with biological and/or chemical activity, characterized in that it comprises as therapeutically active substance in a therapeutic treatment method for the human or animal body, biologically active or physico-chemically active, at least one derivative of diamino-phenothiazine, at least monosubstituted at one of the 1, 2, 3, 4, 5, 6, 7 or 8 positions and having a para and ortho quinonic tautometry, of the general formula (I) or one of its salts,

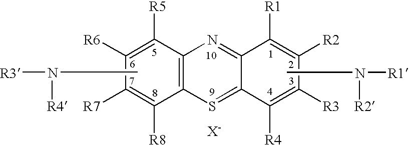

in which

R1, R2, R4, R5, R6, R7, R8 and R1′, R2′, R3′ R4′ are identical or different from each other and represent a hydrogen atom, an aliphatic and/or aromatic and/or cyclic radical, such as an alkyl group, an alkenyl group, an alkynyle group, a hydroxy group, an aryloxy group, a ketone group, an amide group, a carboxy group, a base, etc.,

an inorganic radical such as a halogen atom, an alkaline earth, a metallic atom and

X is a mineral or organic anion.

The term “halogen atom” used in the definition of R1′, R2′, R3′, R4′ and R1, R2, R4, R5, R6, R7, R8 means an atom of chlorine, bromine, fluorine, iodine, etc.

Preferably, an aliphatic, aromatic or cyclic radical is for example a radical comprising 1 to 6 carbon atoms, a fatty acid comprising 10 to 18 carbon atoms, etc.

According to a first preferred embodiment of the invention, the derivatives according to the invention are at least bi-substituted such as at 2-4, 2-5, 2-6, 4-5, 1-4, 1-5, 1-6, 2-8, 2-5, 2-6 or 2-4, as well as all the other possible permutations.

According to a second embodiment of the invention, the derivatives according to the invention are tri-substituted such as at 2-4-5 or 2-4-8, as well as all the other possible permutations.

According to a third embodiment of the invention, the derivatives according to the invention are quadra-substituted such as at 2-3-6-8 or at 2-4-5-8, as well as all the other possible permutations.

Preferably, the derivatives according to the present invention have excellent activity relative to methylene blue, and to its active derivatives. The products thus synthesized are seen particularly to be two to five times more active than the latter, in vitro and in vivo.

The derivatives according to the invention are chemically transformed, according to complex synthesis and purification techniques, forming a completely original series of phenothiazines.

These derivatives more or less have the properties of methylene blue, are hydrosoluble, active by the enteral and parenteral route (IV, SC, etc.) and they never give rise to acute or chronic toxicity nor undesirable effects, at doses comprised between 0.05 mg/Kg and 40 mg/Kg.

The derivatives according to the invention can constitute

a terminal inhibitor of immunologic and immunopathegenic reactions

anti-bacterial agents, anti-viral agents and anti-parasitic agents;

biochemical, biological and physical chemical reagents used as markers, tracers, catalysts, inhibitors, activators

bacteriological, virological, molecular biological, genetic reagents with direct and indirect effect on the nucleic acid structures and strict oxidative metabolism, or all other respiratory systems with the transport of electrons and with gradients;

radio protector in the course of irradiation that is deliberate or accidental of organisms and/or of structures and/or of liquids, biological fluids, chemicals, waters, or agro-food, cosmetic, sunscreen, protector against Liv or gamma radiations;

inhibiting agent for the production of cytopathogenic free radicals due to anti-neoplastic, cytokine treatments or other general toxicities

active substance having anti-rejection activity in the course of organ transplants.

The compositions according to the invention have physico-chemical and biological activities whose potential effects are applicable in the field of agro-food, biology and physical chemistry, and to pathologies often encountered both in human and in veterinary medicine.

The compositions according to the invention are in particular adapted for:

Treatment of afflictions arising from the anti-Tumor Necrosis Factor action (TNFs), anti-interleukines (Ils) pro-inflammatory or not, anti-interferon(s) (INFs).

Treatment of afflictions related to anti-oxidant and anti-free radical action, preferential and specific, on radicals derived from nitrogen,

Treatment of afflictions, such as palladism, arising from the direct and indirect anti-parasitic action on, particularly the genus Plasmodium and Babesia, all the hematozoaires, Toxoplasma, Trypasosoma, Onchocerca, Filaire, Leishmanies, Iematodes, Plathelminthes and Nemathelminthes.

Treatment of afflictions arising from anti-shock, endotoxic, exotoxic, anaphylactic, food, Gram negative and positive bacterial actions.

Treatment of afflictions arising from the action on maladies with an acute or chronic inflammatory action arising or not from research.

Treatment of dysimmune afflictions

Treatment of afflictions arising from the anti-methemoglobin action connected to intoxication with any methemoglobin agents.

Treatment of cerebral and medulary contusions

Treatment of tissue or traumatic reactions, mechanical and thermal, produced or accidental.

Treatment of opportunistic infections and allergic manifestations acquired or congenital immunodeficient conditions.

Treatment of viral and retroviral infections.

Inactivation—disinfection of mushroom virus bacteria, amoebic, parasitic, etc., in agro-food fluids, reactions for treatment of water to render it potable and purified by slow and/or rapid filtration, flocculation decantation, for beverages, etc.

Biochemical, biological, and physical chemical reagents (markers, tracers, catalysts, inhibitors, activators, etc.).

Bacteriological, virological, molecular biological, genetic reagents with direct and indirect effect on the nucleic acid structures and strict oxidative metabolism or any other respiratory systems with electron transport and/or gradients.

Radioprotective effect in the course of irradiation deliberate or accidental of organisms and/or of structures and/or of liquids, biological fluids, chemicals, waters or agri-food, in cosmetics, sunscreens, protection against UV and gamma radiations.

Inhibiting effect on the production of cytopathogenic free radicals due to anti-neoplastic, cytokenic and other general toxic treatments.

Anti-rejection effect on transplanted organs.

Studies have been carried out both in vitro and in vivo on plasmodium falciparum, but also on Babesia canis and on an animal model in endotoxic shock condition produced in mice, sensitivization by Bacillus of Calmette and Guerin (BCG), then injection of Lipopolysaccharides (LPS) extracts of Gram negative bacteria.

It has thus been observed that:

In vivo, the derivatives of the compositions according to the invention are 100% active on declared neurobabesiosis, which is normally incurable with conventional treatments.

In vitro, they totally inhibit the cultures of Plasmodium falciparum and Babesia canis at concentrations of about 10 ppm.

In vitro, the hematoses treated by our products at the same concentrations, rinsed three times, are totally refractory to the culture of the two hematozoaires, whilst remaining perfectly functional.

In viva, the derivatives of the compositions according to the invention are active to the extent of more than 85% on endotoxic shock arising in mice (LPS-BCG), which induces 100% mortality in controls. The effective doses are from 0.5 mg/Kg to 50 mg/Kg. The products are perfectly tolerated and have not given rise to any side effects. Apart from several anti-TNF monoclones, extremely troublesome, and whose activities are very moderate, no actual product can protect animals subjected to shock to this extent. The dosage of the cytokines and other pro-inflammatory agents in protected treated animals causes a very great increase of these products, more than twice the lethal dose.

It is thus possible to conclude that the derivatives and compositions according to the invention do not in any way hinder the normal reaction of endotoxic shock, but that they specifically block the terminal effects of this immunological and acute inflammatory response, namely, they form a complex with the cytokines, and finally their action is doubled.

The retentivity of these derivatives is such that the quantities of circulating molecules are capable of giving false reading of the dosage of TNF by its cytotoxic activity on L 929 cells. The animals protected by these molecules are capable of supporting twice the lethal dose of TNF. Another hypothesis is that the derivatives according to the present invention form a non-functional complex with TNF and the other cytokines, which complex is capable of being recognized by radioimmunology or ELISA type methods.

For these therapeutic purposes, the compositions comprising the derivatives and their salts, alone or in combination with other active principles, are administered by injectable, parenteral, buccal, local route in the form of pharmaceutical compositions adapted to the administration route. The compositions comprise derivatives mixed or added or dissolved in all vehicles, devices or inert excipients, non-toxic, pharmaceutically acceptable, of the injectable dissolved type, tablets, capsules, aromatized powders, syrups, effervescent tablets, tablets with an inert matrix, lyocs, implants, transdermal devices, etc., without this list of galenic forms being exhaustive.

There will now be described the invention in greater detail with reference to the following examples, illustrating the invention in a non-limiting manner.

The examples of derivatives given hereafter have for the basic molecule: 7-(dimethylamino)-3-methylamino-3H phenothiazine (azure B) of the formula

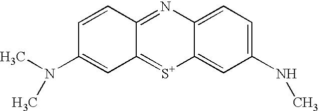

in which R1′ is H and R2′, R3′, R4′ are a methyl group.

### EXAMPLE 1

### 4-5 dimethyl-7-(dimethylamino)-3(methylimino)-3H phenothiazine

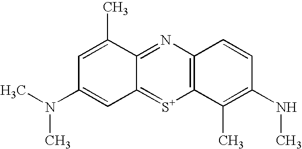

The basic molecule is substituted at 4 and 5 with R4 and R5 being methyl groups.

This compound has the characteristic of being an accentuated electron donor as well as having maximum mesomerie and tautomery whilst having decreased hydrophilicity.

This molecule is a daughter reference molecule whose cationic character is exacerbated without disturbing the weak fundamental dis-equilibrium of its parent molecule.

### EXAMPLE 2

### 5-chloro-4-methyl-7-(dimethylamino )-3-methylimino-3H phenothiazine

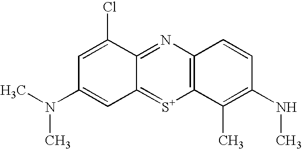

The basic molecule is substituted at 4 and 5, R4 being a methyl group and R5 being a chlorine atom.

The addition of chlorine or another halogen atom considerably increases the half life of the compound. This molecule is more hydrophilic than the reference daughter molecule.

The ionic dis-equilibrium is as follows: chlorine is very attractive of electrons and CH₃ is a strong electron donor and according to the site of substitution the electronic disparity is more diluted.

### EXAMPLE 3

### 4-Chloro-5-methyl-7-(dimethylamino)-3-methylimino-3H phenothiazine

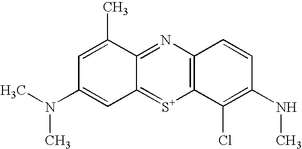

The basic molecule is substituted 4 and 5, R5 being a methyl group and R4 being a chlorine atom.

### EXAMPLE 4

### 4,5-dihydroxy-7-dimethylamino-3 methylimino-3H-phenothiazine

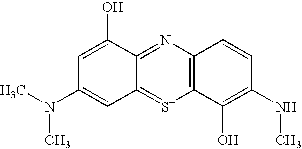

and the formula R4 and R5 are hydroxy groups.

This molecule has a very hydrophilic character and a short half life.

### EXAMPLE 5

### 2, 4-dihydroxy-7-dimethylamino-3-methylimino-3H-phenothiazine

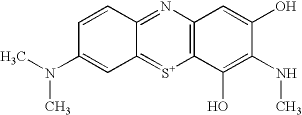

This molecule is di-substituted at 2,4 by hydroxyl groups.

### EXAMPLE 6

### 2-methyl-4-fluoro-7 dimethylamino-3 methylimino-3h phenothiazine

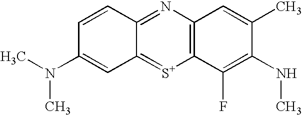

### EXAMPLE 7

### 2-methoxy-5-methyl-7 dimethylamino-3 methylimino-3h phenothiazine

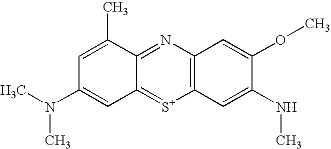

### EXAMPLE 8

### 2-methoxy-5-fluoro-7-dimethylamino-3 methylimino-3h phenothiazine

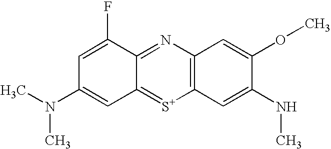

### EXAMPLE 9

### 2-fluoro-5-amino-7- dimethylamino-3 methylimino-3h phenothiazine

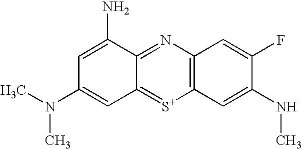

### EXAMPLE 10

### 2-methyl-5-amino-7 dimethylamino-3 methylimino-3h phenothiazine

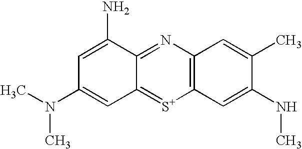

### EXAMPLE 11

### 1-methyl-8-Chloro-7 dimethylamino-3 methylimino-3H phenothiazine

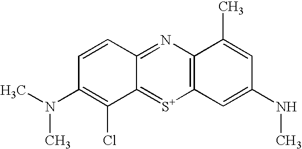

### EXAMPLE 12

### 1-methyl-8-hydroxy-7 dimethylamino-3 methylimino-3 phenothiazine

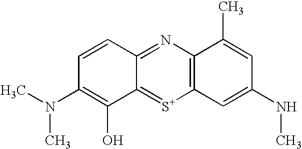

### EXAMPLE 13

### 2-methyl-4-hydroxy-8-Chloro-7 dimethylamino-3 methylimino-3H phenothiazine

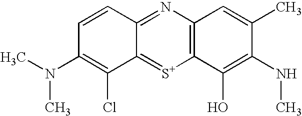

### EXAMPLE 14

### 2,4-dimethyl-8-chloro-7 methylimino-3H phenothiazine

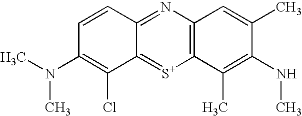

### EXAMPLE 15

### 2, 6-dimethyl-4-hydroxy-8-chloro-7 dimethylamino-3 methylimino-3H phenothiazine

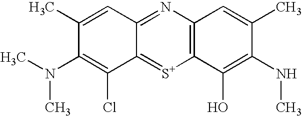

1. Derivative of diamino-phenothiazine, at least monosubstituted at one of the positions 1, 2, 3, 4, 5, 6, 7 or 8 and having a para and ortho quinonic tautometry, of the general formula (I) or one of its salts, 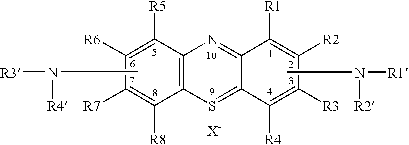
in which
R1, R2, R4, R5, R6, R7, R8 and R1′, R2′, R3′ R4′ are identical or different from each other and represent a hydrogen atom, an aliphatic and/or aromatic and/or cyclic radical, such as an alkyl group, a alkenyl group, an alkynyl group, a hydroxy group, an aryloxy group, a ketone group, an amide group, a carboxy group, a base, an inorganic radical such as a halogen atom, an alkaline earth, a metallic atom and
X is a mineral or organic anion;
excepting the derivatives which are azure A, azure B, azure C, methylene green, the new methylene blue, toluidine blue.

2. Derivative according to claim 1,
characterized in that R1′ is equal to H and R2′, R3′, R4′ are methyl groups.

3. Derivative according to one of claims 1 and 2,
characterized in that it is at least bi-substituted in the positions 1, 2, 3, 4, 5, 6, 7 or 8.

4. Derivative according to one of claims 1 and 2,
characterized in that it is tri-substituted in the positions 1, 2, 3, 4, 5, 6, 7 or 8.

5. Derivative according to one of claims 1 and 2,
characterized in that it is quadri-substituted in the positions 1, 2, 3, 4, 5, 6, 7 or 8.

6. Derivative according to one of claims 1 to 5,
characterized in that it constitutes a terminal inhibitor for the immunological and immunopathogenic reactions.

7. Derivative according to one of claims 1 to 5,
characterized in that it constitutes an anti-bacterial, anti-viral and anti-parasitic agent.

8. Derivative according to one of claims 1 to 5,
characterized in that it constitutes a biochemical, biological and physical chemical reagent such as a marker, tracer, catalyst, inhibitor, activator.

9. Derivative according to one of claims 1 to 5,
characterized in that it constitutes a bacteriological, virological, biological molecular, genetic reagent with direct and indirect effect on the nucleic acid structures and strict oxidative metabolism, or any other respiratory systems with electron transport and gradients.

10. Derivative according to one of claims 1 to 5,
characterized in that it constitutes a radio protector for short irradiations produced or accidental of organism and/or of structures and/or liquids, biological fluids, chemicals, waters or agro-food, as cosmetics, sunscreen, protection against UV or gamma radiations.

11. Derivative according to one of claims 1 to 5,
characterized in that it constitutes an agent for inhibiting the production of free cytopathogenic radicals due to anti-neoplastic treatments, cytokenes or other general toxins.

12. Derivative according to one of claims 1 to 5,
characterized in that it constitutes an active substance having an anti-rejection activity for organ transplants.

13. Composition with biological and/or chemical activity,
characterized in that it comprises as therapeutically active substance in a therapeutic treatment method for the human or animal body, biologically active or physico-chemically active, at least one derivative of diamino-phenothiazine, at least monosubstituted at one of the positions 1, 2, 3, 4, 5, 6, 7 or 8 and having a para and ortho quinonic tautometry, of the general formula (I) or one of its salts, 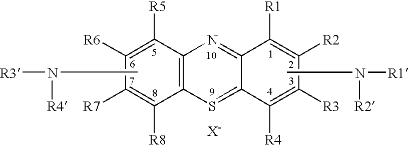
in which
R1, R2, R4, R5, R6, R7, R8 and R1′, R2′, R3′ R4′ are identical or different from each other and represent an atom of hydrogen, an aliphatic and/or aromatic and/or cyclic radical, such as an alkyl group, an alkenyl group, an alkynyle group, a hydroxy group, an aryloxy group, a ketone group, an amide group, a carboxy group, a base, an inorganic radical such as a halogen atom, an alkaline earth, a metallic atom and
X is a mineral or organic anion.

14. Composition with biological and/or chemical activity,
characterized in that it comprises as therapeutically active substance in a method of therapeutic treatment of the human or animal body, biologically active or physico-chemically active, at least one derivative of diamino-phenothiazine, at least monosubstituted at one of the positions 1, 2, 3, 4, 5, 6, 7 or 8 and having a para and ortho quinonic tautometry, of the general formula (I) or one of its salts, 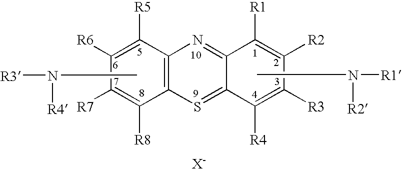
in which
R1, R2, R4, R5, R6, R7, R8 and R1′, R2′, R3′ R4′ are identical or different from each other and represent a hydrogen atom, an aliphatic and/or aromatic and/or cyclic radical, such as an alkyl group, an alkenyl group, an alkynyle group, a hydroxy group, an aryloxy group, a ketone group, an amide group, a carboxy group, a base, an inorganic radical such as a halogen atom, an alkaline earth, a metallic atom; and X is a mineral or organic anion, except derivatives that are azure A, azure B, azure C, methylene green, the new methylene blue, toluidine blue.

15. Composition according to one of claims 13 and 14,
characterized in that the derivative acting as active substance is a terminal inhibitor of the immunological and immunopathogenic reactions.

16. Composition according to one of claims 13 and 14,
characterized in that the derivative as active substance is an anti-bacterial agent, an anti-viral agent and an anti-parasitic agent.

17. Composition according to one of claims 13 and 14,
characterized in that the derivative which is the active substance is a biochemical, biological and physical chemical reagent serving as a marker, tracer, catalyst, inhibitor, activator.

18. Composition according to one of claims 13 and 14,
characterized in that the derivative which is the active substance is a bacteriological, virological, molecular biological, genetic reagent with direct and indirect effect on the nucleic acid structures and strict oxidative metabolism, or any other respiratory systems with electron transport and gradients.

19. Composition according to one of claims 13 and 14,
characterized in that the derivative as the active substance is a radio protector in the course of irradiation that is deliberate or accidental of organisms and/or structures and/or of liquids, biological fluids, chemicals, waters or agri-food, in cosmetic, sunscreen, protection against UV or gamma radiations.

20. Composition according to one of claims 13 and 14,
characterized in that the derivative which is the active substance is an agent inhibiting the production of cytopathogenic free radicals due to anti-neoplastic treatments, cytokines or other general toxins.

21. Composition according to one of claims 13 and 14,
characterized in that the derivative as active substance is an active substance having an anti-rejection activity in the course of organ transplants.

22. Therapeutic composition according to one of claims 13 to 21,
characterized in that it is administrable by injectable, parenteral, buccal and local routes.

23. Use of a composition according to one of claims 13 to 22 for the treatment of afflictions involved in the anti-Tumor Necrosis Factor (TNFs) action, anti-leukine (lle) pro-inflammations or not, anti-interferon (INFs) and all other cytokines.

24. Use of a composition according to one of claims 13 to 22 for the treatment of afflictions involving the anti-oxidant and anti-free radical action, specific and preferential, on nitrite radicals.

25. Use of a composition according to one of claims 13 to 22 for the treatment of afflictions associated with the direct and indirect anti-parasitic action on all the hematozoa, particularly of the genus Plasmodium and Babesia, Toxoplasma, Trypasosoma, Onchocerca, Filairia, Leishmanosis, Nematodes, Plathelminthes and Nemathelminthes.

26. Use of a composition according to one of claims 13 to 22 for the treatment of afflictions arising from the endotoxic, exotoxic, anaphylactic, foodstuff action with Gram negative and positive bacteria.

27. Use of a composition according to one of claims 13 to 22 for the treatment of afflictions arising from the action on maladies with an acute or chronic inflammatory action arising from experiments or not.

28. Use of a composition according to one of claims 13 to 22 for the treatment of dysimmune afflictions.

29. Use of a composition according to one of claims 13 to 22 for the treatment of afflictions arising from the anti-methemoglobinic action connected to intoxications with any methemoglobinic agents.

30. Use of a composition according to one of claims 13 to 22 for the treatment of cerebral and medullary contusions.

31. Use of a composition according to one of claims 13 to 22 for the treatment of tissue reactions to mechanical and thermal trauma, induced or accidental.

32. Use of a composition according to one of claims 13 to 22 for the treatment of opportunistic infections and allergic manifestations taking place under conditions of acquired or congenital immune insufficiency.

33. Use of a composition according to one of claims 13 to 22 for the treatment of viral and retroviral infections.

34. Use of a composition according to one of claims 13 to 22 for the inactivation-disinfection of mushroom virus bacteria, amoeba, reagents for the treatment of water to render it drinkable and purifiable by slow and/or rapid filtration, flocculation decantation.
